(19) United States
(12) Patent Application Publication
(10) Pub. No.: US 2021/0395721 A1
(43) Pub. Date: Dec. 23, 2021

(54) METHODS TO IMPROVE POTENCY OF ELECTROPORATION

(71) Applicants: Codiak BioSciences, Inc., Cambridge, MA (US); MaxCyte, Inc., Gaithersburg, MD (US)

(72) Inventors: Raymond W. BOURDEAU, Watertown, MA (US); Delai CHEN, Cambridge, MA (US); Rane HARRISON, Arlington, MA (US); Kathryn E. GOLDEN, Braintree, MA (US); Douglas E. WILLIAMS, Boston, MA (US); Sergey DZEKUNOV, Germantown, MD (US); Madhusudan PESHWA, Boyds, MD (US)

(73) Assignees: Codiak BioSciences, Inc., Cambridge, MA (US); MaxCyte, Inc., Gaithersburg, MD (US)

(21) Appl. No.: 17/288,719

(22) PCT Filed: Oct. 23, 2019
(86) PCT No.: PCT/US2019/057634

§ 371 Date: Apr. 26, 2021

Related U.S. Application Data
(60) Provisional application No. 62/750,121, filed on Oct. 24, 2018.

(51) Int. Cl. C12N 13/00; C12N 15/87; C12N 15/113

(52) U.S. Cl. CPC C12N 13/00; C12N 15/87; C12N 2310/315; C12N 2310/14; C12N 15/113

(57) ABSTRACT

Compositions and methods for reducing nucleotide oxidation during electroporation, specifically the use of free radical scavengers to reduce electroporation-induced oxidation, are described. Compositions and methods for enhancing transfection efficiency are also described.

### CROSS REFERENCE TO RELATED APPLICATIONS

This PCT application claims the priority benefit of U.S. Provisional Application No. 62/750,121, filed Oct. 24, 2018, which is incorporated herein by reference in its entirety.

### BACKGROUND OF THE INVENTION

### Description of the Related Art

Electroporation is a well-recognized method for delivering molecules, such as polynucleotides, across lipid membranes into mammalian cells, bacterial cells, and membrane vesicles. For example, delivering siRNA and other RNAi molecules using electroporation is an established technique in the field. Current generation siRNA that is being produced for therapeutic purposes often contains chemical modifications that enhance the stability and efficacy of the siRNA. Such modifications include O-methylation of the 2′ position on RNA nucleotides, the introduction of deoxyribonucleotides, and phosphorothioate linkages between the nucleotides. However, whether electroporation alters these nucleotide modifications is unknown. Thus, optimizing electroporation conditions to reduce unintended changes in modified nucleotides is an important need in the field.

### SUMMARY OF THE INVENTION

As described in more detail herein, the addition of free radical scavengers prior to electroporation reduces electroporation-induced alterations in polynucleotides possessing altered nucleotides. In particular, without being bound by theory, the results presented herein suggest free radical scavengers prevent electroporation-induced oxidation of phosphorothioate containing polynucleotides. Also described in more detail herein, the addition of free radical scavengers improves the potency of gene silencing.

Disclosed herein is a method of reducing nucleotide oxidation during electroporation, the method comprising the steps of: 1) providing a composition comprising a) a polynucleotide, wherein the polynucleotide comprises a nucleotide alteration, b) a free radical scavenger, and c) a recipient entity; and 2) electroporating the composition, wherein the free radical scavenger reduces electroporation-induced oxidation of the nucleotide alteration.

In some embodiments, the polynucleotide comprises RNA. In some embodiments, the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof. In some embodiments, the RNA is an siRNA. In some embodiments, the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

In some embodiments, the polynucleotide comprises DNA. In some embodiments, the DNA is selected from the group consisting of: circular plasmids, linear plasmids, vectors, single-stranded DNA, single-stranded oligonucleotides, double-stranded oligonucleotides, a CRISPR system expression vector, and combinations thereof. In some embodiments, the CRISPR system expression vector encodes a CRISPR family enzyme, a gRNA, a crRNA, a tracrRNA, a sgRNA, and combinations thereof.

In some embodiments, the polynucleotide comprises a non-natural nucleic acid. In some embodiments, the non-natural nucleic acid is a morpholino.

In some embodiments, the nucleotide alteration comprises a phosphorothioate internucleotide linkage.

In some embodiments, the free radical scavenger is a reducing agent. In some embodiments, the reducing agent is selected from the group consisting of: L-Methionine, glutathione, L-cysteine, and ascorbic acid, and combinations thereof. In some embodiments, the reducing agent is glutathione.

In some embodiments, the concentration of the free radical scavenger is between 0.1 mM to 100 mM.

In some embodiments, the recipient entity is a lipid-based entity. In some embodiments, the lipid-based entity is selected from the group consisting of: a cell, a vesicle, a tissue, and a lipid-based nanoparticle. In some embodiments, the lipid-based nanoparticle is selected from the group consisting of: a unilamellar liposome, a multilamellar liposome, a nanovesicle, and a lipid preparation. In some embodiments, the vesicle is an extracellular vesicle. In some embodiments, the extracellular vesicle is an exosome. In some embodiments, the cell is selected from a eukaryotic cell or a prokaryotic cell. In some embodiments, the eukaryotic cell is selected from the group consisting of: an animal cell, a fungal cell, and a plant cell. In some embodiments, the animal cell is selected from a vertebrate cell or an invertebrate cell. In some embodiments, the vertebrate cell is a mammalian cell. In some embodiments, the mammalian cell is a human cell. In some embodiments, the cell is selected from the group consisting of: a stem cell, an immune cell, an erythrocyte, a cancer cell, a cultured cell, an immortalized cell, and an isolated cell, and combinations thereof. In some embodiments, the immune cell is selected from the group consisting of: a T cell, a B cell, a macrophage, and a dendritic cell. In some embodiments, the fungal cell is a yeast cell. In some embodiments, the prokaryotic cell is a bacterial cell.

In some embodiments, the recipient entity is a non-lipid entity. In some embodiments, the non-lipid entity is a non-lipid nanostructure.

In some embodiments, the electroporating step is performed in vitro, in vivo, or ex vivo.

In some embodiments, the reduction in oxidation is determined through analyzing a molecular profile of the polynucleotide. In some embodiments, the molecular profile is an anion exchange high-performance liquid chromatography (AEX-HPLC) chromatogram. In some embodiments, the molecular profile is an ion-pairing reversed-phase chromatography (IPRP-HPLC) chromatogram. In some embodiments, the molecular profile is a mass spectrometry spectrum. In some embodiments, the molecular profile of the polynucleotide is shifted toward an unelectroporated polynucleotide relative to a polynucleotide electroporated in the absence of the free radical scavenger.

In some embodiments, the electroporating step comprises a voltage level higher than a viable electroporation voltage level in the absence of the free radical scavenger. In some embodiments, the polynucleotide demonstrates a functional improvement at the voltage level. In some embodiments, the functional improvement is an increased activity of the polynucleotide. In some embodiments, the increased activity of the polynucleotide is an increase in RNA interference. In some embodiments, the increased activity of the polynucleotide is an increase in CRISPR mediated gene editing.

Also described herein is a method of enhancing transfection efficiency, comprising the steps of: 1) providing a composition comprising a) a polynucleotide, wherein the polynucleotide comprises a nucleotide alteration, b) a free radical scavenger, and c) a recipient entity; and 2) electroporating the composition, wherein the free radical scavenger reduces electroporation-induced oxidation of the electroporated polynucleotide.

In some embodiments, the polynucleotide comprises RNA. In some embodiments, the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof.

In some embodiments, the RNA is an siRNA. In some embodiments, the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

In some embodiments, the polynucleotide comprises DNA. In some embodiments, the DNA is selected from the group consisting of: circular plasmids, linear plasmids, vectors, single-stranded DNA, single-stranded oligonucleotides, double-stranded oligonucleotides, a CRISPR system expression vector, and combinations thereof. In some embodiments, the CRISPR system expression vector encodes a CRISPR family enzyme, a gRNA, a crRNA, a tracrRNA, a sgRNA, and combinations thereof.

In some embodiments, the polynucleotide comprises a non-natural nucleic acid. In some embodiments, the non-natural nucleic acid is a morpholino.

In some embodiments, the nucleotide alteration comprises a phosphorothioate internucleotide linkage.

In some embodiments, the free radical scavenger is a reducing agent. In some embodiments, the reducing agent is selected from the group consisting of: L-Methionine, glutathione, L-cysteine, and ascorbic acid, and combinations thereof. In some embodiments, the reducing agent is glutathione.

In some embodiments, the concentration of the free radical scavenger is between 0.1 mM to 100 mM.

In some embodiments, the recipient entity is a lipid-based entity. In some embodiments, the lipid-based entity is selected from the group consisting of: a cell, a vesicle, a tissue, and a lipid-based nanoparticle. In some embodiments, the lipid-based nanoparticle is selected from the group consisting of: a unilamellar liposome, a multilamellar liposome, a nanovesicle, and a lipid preparation. In some embodiments, the vesicle is an extracellular vesicle. In some embodiments, the extracellular vesicle is an exosome. In some embodiments, the cell is selected from a eukaryotic cell or a prokaryotic cell. In some embodiments, the eukaryotic cell is selected from the group consisting of: an animal cell, a fungal cell, and a plant cell. In some embodiments, the animal cell is selected from a vertebrate cell or an invertebrate cell. In some embodiments, the vertebrate cell is a mammalian cell. In some embodiments, the mammalian cell is a human cell. In some embodiments, the cell is selected from the group consisting of: a stem cell, an immune cell, an erythrocyte, a cancer cell, a cultured cell, an immortalized cell, and an isolated cell, and combinations thereof. In some embodiments, the immune cell is selected from the group consisting of: a T cell, a B cell, a macrophage, and a dendritic cell. In some embodiments, the fungal cell is a yeast cell. In some embodiments, the prokaryotic cell is a bacterial cell.

In some embodiments, the recipient entity is a non-lipid entity. In some embodiments, the non-lipid entity is a non-lipid nanostructure.

In some embodiments, the electroporating step is performed in vitro, in vivo, or ex vivo.

In some embodiments, the reduction in oxidation is determined through analyzing a molecular profile of the polynucleotide. In some embodiments, the molecular profile is an anion exchange high-performance liquid chromatography (AEX-HPLC) chromatogram. In some embodiments, the molecular profile is an ion-pairing reversed-phase chromatography (IPRP-HPLC) chromatogram. In some embodiments, the molecular profile is a mass spectrometry spectrum. In some embodiments, the molecular profile of the polynucleotide is shifted toward an unelectroporated polynucleotide relative to a polynucleotide electroporated in the absence of the free radical scavenger.

In some embodiments, the electroporating step comprises a voltage level higher than a viable electroporation voltage level in the absence of the free radical scavenger. In some embodiments, the polynucleotide demonstrates a functional improvement at the voltage level. In some embodiments, the functional improvement is an increased activity of the polynucleotide. In some embodiments, the increased activity of the polynucleotide is an increase in RNA interference. In some embodiments, the increased activity of the polynucleotide is an increase in CRISPR mediated gene editing.

Also described herein is a composition for reducing nucleotide oxidation during electroporation, the composition comprising a) a polynucleotide, wherein the polynucleotide comprises a nucleotide alteration, b) a free radical scavenger, and c) a recipient entity.

In some embodiments, the polynucleotide comprises RNA. In some embodiments, the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof. In some embodiments, the RNA is an siRNA. In some embodiments, the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

In some embodiments, the polynucleotide comprises DNA. In some embodiments, the DNA is selected from the group consisting of: circular plasmids, linear plasmids, vectors, single-stranded DNA, single-stranded oligonucleotides, double-stranded oligonucleotides, a CRISPR system expression vector, and combinations thereof. In some embodiments, the CRISPR system expression vector encodes a CRISPR family enzyme, a gRNA, a crRNA, a tracrRNA, a sgRNA, and combinations thereof.

In some embodiments, the polynucleotide comprises a non-natural nucleic acid. In some embodiments, the non-natural nucleic acid is a morpholino.

In some embodiments, the nucleotide alteration comprises a phosphorothioate internucleotide linkage. In some embodiments, the free radical scavenger is a reducing agent.

In some embodimentss, the reducing agent is selected from the group consisting of: L-Methionine, glutathione, L-cysteine, and ascorbic acid, and combinations thereof. In some embodiments, the reducing agent is glutathione.

In some embodiments, the concentration of the free radical scavenger is between 0.1 mM to 100 mM.

In some embodiments, the recipient entity is a lipid-based entity.

In some embodiments, the lipid-based entity is selected from the group consisting of: a cell, a vesicle, a tissue, and a lipid-based nanoparticle. In some embodiments, the lipid-based nanoparticle is selected from the group consisting of: a unilamellar liposome, a multilamellar liposome, a nanovesicle, and a lipid preparation. In some embodiments, the vesicle is an extracellular vesicle. In some embodiments, the extracellular vesicle is an exosome. In some embodiments, the cell is selected from a eukaryotic cell or a prokaryotic cell. In some embodiments, the eukaryotic cell is selected from the group consisting of: an animal cell, a fungal cell, and a plant cell. In some embodiments, the animal cell is selected from a vertebrate cell or an invertebrate cell. In some embodiments, the vertebrate cell is a mammalian cell. In some embodiments, the mammalian cell is a human cell. In some embodiments, the cell is selected from the group consisting of: a stem cell, an immune cell, an erythrocyte, a cancer cell, a cultured cell, an immortalized cell, and an isolated cell, and combinations thereof. In some embodiments, the immune cell is selected from the group consisting of: a T cell, a B cell, a macrophage, and a dendritic cell. In some embodiments, the fungal cell is a yeast cell. In some embodiments, the prokaryotic cell is a bacterial cell.

In some embodiments, the recipient entity is a non-lipid entity. In some embodiments, the non-lipid entity is a non-lipid nanostructure.

Also described herein is a method of reducing nucleotide oxidation during electroporation, the method comprising the steps of: 1) providing a composition comprising any of the compositions described herein; and 2) electroporating the composition, wherein the free radical scavenger reduces electroporation-induced oxidation of the nucleotide alteration.

Also described herein method of enhancing transfection efficiency, the method comprising the steps of: 1) providing a composition comprising any of the compositions described herein; and 2) electroporating the composition, wherein the free radical scavenger reduces electroporation-induced oxidation of the electroporated polynucleotide.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates anion exchange chromatography profiles demonstrating modified siRNA (XD-08318) is altered following electroporation. The electroporated samples (two left peaks) are shifted compared to the unelectroporated sample (right peak).

FIG. 2 illustrates the general nucleotide modifications in siRNA XD-08318.

FIG. 3 illustrates anion exchange chromatography profiles demonstrating unmodified siRNA (KRAS G12D), i.e., siRNA containing only native ribonucleotide chemistries, is unaltered during electroporation. The electroporated sample and the unelectroporated sample profiles overlap.

FIG. 4 illustrates anion exchange chromatography profiles demonstrating increased electroporation-induced alterations under stronger pulse-strength electroporation conditions. The strongest pulse-strength electroporation condition (“PC66” left most peaks) are shifted furthest to the left compared to weakest pulse-strength electroporation condition (“PC11” middle peaks) and the unelectroporated sample (“mix control” right peak).

FIG. 5 illustrates anion exchange chromatography profiles demonstrating electroporated modified siRNA resembles oxidized siRNA. The electroporated and oxidized samples (left peaks) are shifted compared to the control sample.

FIG. 6 illustrates anion exchange chromatography profiles demonstrating the free radical scavenger L-methionine reduces electroporation-induced alterations in modified siRNA (XD-08318). Samples with the free radical scavenger added (“0.1 mM-5 mM” right peaks) are less shifted compared to the sample without free radical scavengers added (left peak).

FIG. 7 illustrates anion exchange chromatography profiles demonstrating the free radical scavenger glutathione reduces electroporation-induced alterations in modified siRNA (XD-08318). Samples with the free radical scavenger added (“0.1 mM-5 mM” right peaks) are less shifted compared to the sample without free radical scavengers added (left peak).

FIG. 8 illustrates anion exchange chromatography profiles demonstrating the free radical scavenger ascorbate reduces electroporation-induced alterations in modified siRNA (XD-08318) under specific electroporation conditions. Samples with the free radical scavenger added (“0.1 mM-5 mM” right peaks) are less shifted compared to the sample without free radical scavengers added (left peak).

FIG. 9 illustrates anion exchange chromatography profiles demonstrating the free radical scavenger L-methionine reduces electroporation-induced alterations in modified siRNA (XD-08318) under specific electroporation conditions. Samples with the free radical scavenger added (“0.1 mM-5 mM” right peaks) are less shifted compared to the sample without free radical scavengers added (left peak).

FIG. 10 illustrates anion exchange chromatography profiles demonstrating the free radical scavenger cysteine reduces electroporation-induced alterations in modified siRNA (XD-08318) under specific electroporation conditions using a high strength, low frequency pulse (PC63). Samples with the free radical scavenger added (“0.1 mM-5 mM” right peaks) are less shifted compared to the sample without free radical scavengers added (left peak).

FIG. 11 illustrates anion exchange chromatography profiles demonstrating the free radical scavenger cysteine reduces electroporation-induced alterations in modified siRNA (XD-08318) under specific electroporation conditions using a high strength, high frequency pulse (PC66). Samples with the free radical scavenger added (“0.1 mM-5 mM” right peaks) are less shifted compared to the sample without free radical scavengers added (left peak).

FIG. 12 illustrates anion exchange chromatography profiles demonstrating the free radical scavenger glutathione reduces electroporation-induced alterations in modified siRNA (XD-08318) using the Neon electroporation system. Samples with the free radical scavenger added (“0.1 mM-5 mM” right peaks) are less shifted compared to the sample without free radical scavengers added (left peak).

FIG. 13 illustrates anion exchange chromatography profiles demonstrating the free radical scavenger glutathione reduces electroporation-induced alterations in modified siRNA (XD-08318) using the Bio-Rad electroporation system. Samples with the free radical scavenger added (“0.1 mM-5 mM” left peaks) are less shifted compared to the sample without free radical scavengers added (right peak). The profile also demonstrates the electroporation-induced shift is different between electroporation systems.

FIG. 14 illustrates modified siRNA potency increases when free radical scavengers are added during electroporation into exosomes. Normalized gene expression is presented for various conditions. Modified siRNA electroporated in the presence of ascorbic acid (#9) or L-methionine (#10) demonstrated increased knock-down compared to electroporation in the absence of free radical scavengers (#4-8).

FIG. 15 illustrates that in the presence of methionine, a broad range of electroporation conditions are suitable for loading siRNA into exosomes and allowing for knockdown of a target gene.

FIG. 16 illustrates increasing pulse-strength electroporation conditions (PC-43 vs PC-66) in the presence of glutathione and the corresponding increase in the number of siRNA molecules per exosome.

FIG. 17 illustrates increased cellular phenotypic outcomes associated with increased siRNA potency under strong electroporation conditions in the presence of free radical scavengers.

### DETAILED DESCRIPTION

### Advantages and Utility

Briefly, and as described in more detail below, is an improved method of electroporating modified nucleotides. The results presented herein demonstrate electroporation of modified polynucleotides results in an alteration of the polynucleotides. The improved method adds free radical scavengers prior to electroporation to reduce said alterations. The methods described herein are a significant improvement over the state of the art and fulfill an unmet need in the field of polynucleotide electroporation.

### Definitions

Terms used in the claims and specification are defined as set forth below unless otherwise specified.

As used herein, “polynucleotides” refer to a linear polymer comprised of nucleotides including, but not limited to, deoxyribonucleic acids (DNA), ribonucleic acids (RNA), and non-natural nucleic acids.

As used herein, “RNAi” or “RNA interference” refers to the use of RNA based polynucleotides to alter gene expression, generally through targeting RNA molecules for cleavage and degradation, or inhibiting the target RNA's interaction with downstream cellular pathways, such as translational machinery.

As used herein, “free radicals” refer to unpaired electrons or molecules which contain unpaired electrons.

As used herein, “electroporation” refers to the method of applying an electrical field to a recipient entity to transiently permeabilize the outer membrane or shell of the entity, allowing for internalization of a cargo into the entity's interior compartment.

As used herein, a “recipient entity” is any structure that can receive a cargo upon electroporation.

As used herein, a “liposome” is a generic term encompassing a variety of single and multilamellar lipid vehicles formed by the generation of enclosed lipid bilayers or aggregates.

As used herein, the term “extracellular vesicle” refers to a cell-derived vesicle comprising a membrane that encloses an internal space.

As used herein, the term “nanovesicle” refers to a cell-derived small (between 20-250 nm in diameter, more preferably 30-150 nm in diameter) vesicle comprising a membrane that encloses an internal space, and which is generated from a cell by direct or indirect manipulation such that the nanovesicle would not be produced by the producer cell without said manipulation.

As used herein, the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane.

It must be noted that, as used in the specification and the appended claims, the singular forms “a,” “an” and “the” include plural referents unless the context clearly dictates otherwise.

### Methods of the Invention

Described herein are methods for the reducing nucleotide oxidation during electroporation.

In a first aspect of the invention, the method comprises the steps of 1) providing a composition comprising a) a polynucleotide, wherein the polynucleotide comprises a nucleotide alteration, b) a free radical scavenger, and c) a recipient entity; and 2) electroporating the composition, wherein the free radical scavenger reduces electroporation-induced oxidation of the nucleotide alteration.

### I. Polynucleotides

As used herein, “polynucleotides” refer to a linear polymer comprised of nucleotides including, but not limited to, deoxyribonucleic acids (DNA), ribonucleic acids (RNA), and non-natural nucleic acids. In a variety of embodiments, DNA and RNA are comprised of nucleobases (e.g., cytosine, guanine, adenine, thymine, and uracil), ribose (RNA) or deoxyribose (DNA) sugars, and phosphate groups.

In a variety of embodiments, polynucleotides are altered (used herein, “altered” and “modified” may be used interchangeably). Various nucleotide alterations, and the methods to produce polynucleotides containing such alterations, are well-known to those skilled in the art. For example, in particular embodiments, alterations comprise the addition of non-nucleotide material, including internally (at one or more nucleotides) and/or to the end(s) of the polynucleotides. In certain embodiments, polynucleotides have one alteration. In other embodiments, polynucleotides have more than one alteration. In particular embodiments, polynucleotides have more than one type of alteration. In specific embodiments, the types of alterations include, but are not limited to, a 3′-hydroxyl group, 2′-O-methyl ribonucleotides, 2′-deoxy-2′-fluoro ribonucleotides, “universal base” nucleotides, 5-C-methyl nucleotides, one or more modified internucleotide linkages, and inverted deoxy abasic residue incorporation, and as described in further detail in U.S. Application Publication 2004/0019001 and U.S. Pat. No. 6,673,611 (each of which is incorporated by reference in its entirety). In other embodiments, alterations comprise the addition of non-natural nucleic acids including, but not limited to, peptide nucleic acid (PNA), locked nucleic acid (LNA), glycol nucleic acid (GNA), threose nucleic acid (TNA), and phosphorodiamidate morpholino oligomer (PMO or “morpholino”). Non-natural nucleic acids are described in further detail in U.S. Pat. Nos. 5,185,444, 5,539,082, 6,670,461, International Application WO 1998/039352, and U.S. Application Pub. No. 2013/0156849.

In specific embodiments, the nucleotides are linked together via a phosphodiester bond. In particular embodiments, the nucleotides are altered such that one or more of the phosphodiester bonds are replaced by a modified internucleotide linkage, for example, phosphorothioate, phosphorodithioate, or other modified internucleotide linkages known in the art. In a particular embodiment, the modified internucleotide linkage is a phosphorothioate linkage. Phosphorothioate linkages are well-established in the art for the purposes of reducing oligonucleotide degradation, such as nuclease mediated degradation and hydrolysis. In another particular embodiment, the polynucleotide comprises one or more modified internucleotide linkages in combination with other types of alterations.

In a variety of embodiments, polynucleotides are single-stranded. In other embodiments, polynucleotides are double-stranded. In certain embodiments, double-stranded polynucleotides comprises overhangs that are not base paired to a complementary strand. In particular embodiments, double-stranded polynucleotides comprises two strands that base pair with 100% complementarity. In other embodiments, double-stranded polynucleotides comprises two strands that contain one or more mismatches.

In various embodiments, polynucleotides are linear. In other embodiments, polynucleotides are circular. In certain embodiments, polynucleotides self-hybridize. In particular embodiments, self-hybridized polynucleotides form an unpaired stem-loop or hairpin.

In a variety of embodiments, polynucleotides are synthesized. In other embodiments, polynucleotides are amplified, such as by polymerase chain reaction (PCR). In still other embodiments, polynucleotides are isolated from biological entities (examples are described in more detail in Section III).

Exemplary RNA polynucleotides of the present invention include, but are not limited to, siRNAs, miRNAs, antisense oligonucleotides, shRNAs, double-stranded RNAs, RNA oligonucleotides, mRNAs, or combinations thereof. In a particular embodiment, the RNA polynucleotide is an siRNA.

Exemplary DNA polynucleotides of the present invention include, but are not limited to, circular plasmids, linear plasmids, vectors, single-stranded DNA, single-stranded oligonucleotides, and double-stranded oligonucleotides.

In certain embodiments, the polynucleotide is a Clustered Regularly Interspaced Short Palindromic Repeats (CRISPR) system polynucleotide. In specific embodiments, CRISPR polynucleotides include, but are not limited to, a guide RNA (gRNA), a CRISPR RNA (crRNA), a trans-activating CRISPR RNA (tracrRNA), a single-guide crRNA and tracrRNA fusion (sgRNA), an expression vector encoding a CRISPR family nuclease, an expression vector encoding a gRNA, an expression vector encoding a crRNA, an expression vector encoding a tracrRNA, an expression vector encoding an sgRNA, or a homology repair template.

### II. Free Radical Scavengers

As used herein, “free radicals” refer to unpaired electrons or molecules which contain unpaired electrons. Free radicals are generally highly chemically reactive and can catalyze redox reactions that can propagate. For example, a free radical may take an electron from another molecule, referred to as oxidizing the molecule. In turn, the oxidized molecule itself can take an electron from yet another molecule, generating a chain reaction. Free radicals may form through the process of homolysis, where a relatively large amount of energy breaks a chemical bond to form two radicals. Without being bound by theory, electroporation may provide the energy required for homolysis. Free radicals can oxidize a variety of biological molecules, including polynucleotides, lipids, fatty acids, and proteins. Notable biological free radicals include, but are not limited to, superoxide and nitric oxide.

In the present invention, electroporation can result in damaging or otherwise altering the properties of an electroporated polynucleotide. In specific embodiments, polynucleotide oxidation damages or otherwise alters the properties of the polynucleotide. In other embodiments, free radicals may oxidize polynucleotide modifications that damage or otherwise alter the properties of the polynucleotide. Examples of polynucleotide modifications are described in greater detail in Section I. In various embodiments, electroporation can result in electroporation-induced oxidation of nucleotide alterations.

In certain embodiments, the altered properties of the electroporated polynucleotide are determined through analyzing a molecular profile of the polynucleotide. In various embodiments, the molecular profile of the electroporated polynucleotide is shifted relative to an unelectroporated polynucleotide, representing an altered property of the electroporated polynucleotide. In specific embodiments, the altered property is electroporation-induced oxidation of the electroporated polynucleotide. In particular embodiments, the altered properties of the electroporated polynucleotide are determined through analyzing an anion exchange high-performance liquid chromatography (AEX-HPLC) chromatogram. In another particular embodiment, the altered properties of the electroporated polynucleotide are determined through analyzing an ion-pairing reversed-phase chromatography (IPRP-HPLC) chromatogram. In another embodiment, the altered properties of the electroporated polynucleotide are determined through analyzing a mass spectrometry spectrum.

In the present invention, “free radical scavengers” refer to molecules that chemically react with free radicals, generally resulting in reaction products that are less reactive. In various embodiments, free radical scavengers are reducing agents. Reducing agents donate an electron to free radicals such that the free radical is reduced (gains an electron) and the reducing agent is oxidized (loses an electron). In certain embodiments, the reducing agent acts as an antioxidant. In general, an antioxidant refers to a molecule that in its oxidized form is relatively stable. Thus, antioxidants can terminate redox chain reactions since both reaction products, the reduced free radical and the oxidized antioxidant, are relatively stable.

Numerous reducing agents that can act as free radical scavengers and antioxidants exist and are contemplated by the current invention. Many reducing agents are produced naturally. In specific embodiments, such reducing agents include, but are not limited to, L-Methionine, glutathione, L-cysteine, ascorbic acid, uric acid, α-tocopherol (Vitamin E), lipoic acid, β-carotene, retinol (Vitamin A), and ubiquinol. In a particular embodiment, the reducing agent is glutathione.

Free radical scavengers can be used in the present invention at various concentrations. The optimal concentration for the free radical scavenger will depend on various aspects, such as properties of the free radical scavenger itself, electroporation conditions (see Section IV), properties of the polynucleotide, properties of any nucleotide alteration, viability of a recipient entity (see Section III). In various embodiments, the concentration of the free radical scavenger is at least 0.1 mM, at least 0.5 mM, at least 1 mM, at least 5 mM, at least 10 mM, at least 50 mM, or at least 100 mM. In certain embodiments, the concentration of the free radical scavenger is between 0.1-100 mM. In some embodiments, the concentration of the free radical scavenger is between 0.1-0.5 mM, between 0.5-1 mM, between 1-5 mM, between 5-10 mM, between 10-50 mM, or between 50-100 mM. In specific embodiments, the concentration of the free radical scavenger is 0.1 mM, 0.5 mM, 1 mM, 5 mM, 10 mM, 50 mM, or 100 mM.

In certain embodiments, the reduction in the altered properties of the electroporated polynucleotide are determined through analyzing a molecular profile of the polynucleotide. In various embodiments, the molecular profile of the polynucleotide is shifted toward an unelectroporated polynucleotide relative to a polynucleotide electroporated in the absence of the free radical scavenger, representing a reduction in the altered properties of the electroporated polynucleotide. In particular embodiments, the reduction in the altered properties of the electroporated polynucleotide are determined through analyzing an anion exchange high-performance liquid chromatography (AEX-HPLC) chromatogram. In another particular embodiment, the reduction in the altered properties of the electroporated polynucleotide are determined through analyzing an ion-pairing reversed-phase chromatography (IPRP-HPLC) chromatogram. In another embodiment, the reduction in the altered properties of the electroporated polynucleotide are determined through analyzing a mass spectrometry spectrum.

In certain embodiments, the reduction in electroporation-induced oxidation of the electroporated polynucleotide are determined through analyzing a molecular profile of the polynucleotide. In various embodiments, the molecular profile of the polynucleotide is shifted toward an unelectroporated polynucleotide relative to a polynucleotide electroporated in the absence of the free radical scavenger, representing a reduction in the electroporation-induced oxidation of the electroporated polynucleotide. In particular embodiments, the reduction in the altered properties of the electroporated polynucleotide are determined through analyzing an anion exchange high-performance liquid chromatography (AEX-HPLC) chromatogram. In another particular embodiment, the reduction in electroporation-induced oxidation of the electroporated polynucleotide are determined through analyzing an ion-pairing reversed-phase chromatography (IPRP-HPLC) chromatogram. In another embodiment, the reduction in the electroporation-induced oxidation of the electroporated polynucleotide are determined through analyzing a mass spectrometry spectrum.

### III. Recipient Entities

As used herein, a “recipient entity” is any structure that can receive a cargo upon electroporation. In various embodiments, the recipient entity is a lipid-based entity. In general, a lipid-based entity refers to a structure composed of an outer lipid membrane enveloping an internal compartment. In various embodiments, the lipid-based entity includes, but is not limited to, a lipid-based nanoparticle, a vesicle, a cell, or a tissue.

In various embodiments, the lipid-based nanoparticle includes, but is not limited to, a unilamellar liposome, a multilamellar liposome, a nanovesicle, and a lipid preparation. As used herein, a “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. In specific embodiments, liposomes include unilamellar liposomes, multilamellar liposomes, and multivesicular liposomes. In other embodiments, liposomes may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer. In specific embodiments, a cargo, such as a polypeptide, a nucleic acid, or a small molecule drug, may be encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

Preparations of liposomes are described in further detail in WO 2016/201323, which is hereby incorporated by reference in its entirety

As used herein, the term “nanovesicle” refers to a cell-derived small (between 20-250 nm in diameter, more preferably 30-150 nm in diameter) vesicle comprising a membrane that encloses an internal space, and which is generated from a cell by direct or indirect manipulation such that the nanovesicle would not be produced by the producer cell without said manipulation. Appropriate manipulations of a producer cell include, but are not limited to, serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof

In various embodiments, the lipid-based entity is a vesicle. In specific embodiments, the vesicle is an extracellular vesicle. As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and may comprise various macromolecular cargo either within the internal space, displayed on the external surface of the extracellular vesicle, and/or spanning the membrane. Said cargo may comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles may be derived from a living or dead organism, explanted tissues or organs, and/or cultured cells.

In a particular embodiment, the extracellular vesicle is an exosome. As used herein, the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. Generally, production of exosomes does not result in the destruction of the producer cell. The exosome comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof.

Exosomes and preparation of exosomes are described in further detail in WO 2016/201323, which is hereby incorporated by reference in its entirety.

In various embodiments, the lipid-based entity is a cell. In specific embodiments, the cell can be eukaryotic or prokaryotic. In a variety of embodiments, a eukaryotic cell includes, but is not limited to, an animal cell, a fungal cell, or a plant cell. In specific embodiments, the animal cell is an invertebrate or vertebrate cell. In one embodiment, the vertebrate cell is a mammalian cell. In particular embodiment, the mammalian cell is a human cell. In specific embodiments, the lipid-based entity is a platelet.

In a series of embodiments, a cell includes, but is not limited to, a stem cell, an immune cell, an erythrocyte, a cancer cell, a cultured cell, an immortalized cell, an isolated cell, or a combination of the above. For example, an immune cell can also be a cancer cell, a cultured cell, an immortalized cell, and/or an isolated cell.

In a specific series of embodiments, an immune cell includes, but is not limited to, a T cell, a B cell, a macrophage, and a dendritic cell.

In one embodiment, a fungal cell is a yeast cell. In another embodiment, a prokaryotic cell is a bacterial cell.

In another series of embodiments, the recipient entity is a non-lipid entity. In a specific embodiment, the non-lipid entity is a non-lipid nanostructure.

### IV. Electroporation

In the present invention, electroporation is used to deliver polynucleotides to recipient entities. As used herein, “electroporation” refers to the method of applying an electrical field to a recipient entity to transiently permeabilize the outer membrane or shell of the entity, allowing for internalization of a cargo into the entity's interior compartment. Electroporation techniques are well-known to those skilled in the art. In an illustrative example, a large number of cells within a solution containing a cargo of interest are placed between two electrodes. A set voltage is transiently applied to the cells and the lipid membrane of the cells is disrupted, i.e., permeabilized, allowing the cargo to enter the cytoplasm of the cell. Importantly, at least a portion of the cells that internalized the cargo remain viable.

Electroporation conditions (e.g., voltage, time, capacitance, number of cells, concentration of cargo, volume, cuvette length, pulse type, pulse length, electroporation solution composition, recovery conditions, etc.) vary depending on several factors including, but not limited to, the type of cell or other recipient entity, the cargo to be delivered, the efficiency of internalization desired, and the viability desired. Optimization of such criteria are within the scope of those skilled in the art. A variety of electroporation devices and protocols can be used to carry out the present invention. Examples include, but are not limited to, MaxCyte° Flow Electroporation™, Neon® Transfection System, Bio-Rad® electroporation systems, and Lonza® Nucleofector™ systems.

Pulses can be square wave or exponential decay pulse models.

Cuvette length can be between 0.1-0.4 cm, such as 0.1 cm, 0.2 cm, 0.3 cm, or 0.4 cm.

Volume can be between 10-200 μL, such as 10 μL, 20 μL, 30 μL, 40 μL, 50 μL, 60 μL, 70 μL, 80 μL, 90 μL, 110 μL, 120 μL, 130 μL, 140 μL, 150 μL, 160 μL, 170 μL, 180 μL, 190 μL. Volume can be 100 μL.

Exemplary voltages for mammalian cells include, but are not limited to, 100-200 V, such as 100 V, 110 V, 120 V, 130 V, 140 V, 150 V, 155 V, 160 V, 170 V, 180 V, 190 V, or 200 V. Exemplary voltages for bacterial cells include, but are not limited to, 1000-3000 V, such as 1000 V, 1100 V, 1200 V, 1300 V, 1400 V, 1500 V, 1600 V, 1700 V, 1800 V, 1900 V, 2000 V, 2100 V, 2200 V, 2300 V, 2400 V, 2500 V, 2600 V, 2700 V, 2800 V, 2900 V, or 3000 V. Exemplary voltages for fungal cells include, but are not limited to, 1000-3000 V, such as 1000 V, 1100 V, 1200 V, 1300 V, 1400 V, 1500 V, 1600 V, 1700 V, 1800 V, 1900 V, 2000 V, 2100 V, 2200 V, 2300 V, 2400 V, 2500 V, 2600 V, 2700 V, 2800 V, 2900 V, or 3000 V.

Exemplary capacitance for mammalian cells in exponential decay pulse models include, but are not limited to, 100-2000 μF. Exemplary capacitance for mammalian cells in exponential decay pulse models can be 500 μF. Exemplary capacitance for mammalian cells in exponential decay pulse models can be 1000 μF. Exemplary capacitance for bacterial cells in exponential decay pulse models include, but are not limited to, 10-100 μF. Exemplary capacitance for bacterial cells in exponential decay pulse models can be 50 μF. Exemplary capacitance for bacterial cells in exponential decay pulse models can be 25 μF. Exemplary capacitance for yeast cells in exponential decay pulse models include, but are not limited to, 10-100 μF. Exemplary capacitance for yeast cells in exponential decay pulse models can be 10 μF. Exemplary capacitance for yeast cells in exponential decay pulse models can be 25 μF.

Exemplary pulse lengths for mammalian cells in square wave pulse models include, but are not limited to, 5-50 msec. Exemplary pulse lengths for mammalian cells in square wave pulse models can be 10 msec. Exemplary pulse lengths for mammalian cells in square wave pulse models can be 15 msec. Exemplary pulse lengths for mammalian cells in square wave pulse models can be 20 msec. Exemplary pulse lengths for mammalian cells in square wave pulse models can be 25 msec.

In a variety of embodiments, electroporation is performed in vitro, in vivo, or ex vivo.

In certain embodiments of the invention, the presence of free radical scavengers allows for a greater possible voltage (also referred to as pulse strength) to be used during electroporation. In specific embodiments, the greater voltage allows for improved transfection efficiency and/or a functional improvement in the delivered cargo. In a particular embodiment, the electroporated polynucleotide demonstrates a functional improvement.

### V. RNAi

As used herein, “RNAi” or “RNA interference” refers to the use of RNA based polynucleotides to alter gene expression, generally through targeting RNA molecules for cleavage and degradation, or inhibiting the target RNA's interaction with downstream cellular pathways, such as translational machinery. A variety of RNA polynucleotides can lead to RNAi. In some aspects, the RNAi polynucleotide include, but are not limited to, double-stranded RNA (dsRNA), small-interfering RNA (siRNA), short-hairpin (shRNA), microRNA (miRNA), and pre-miRNA. In a particular embodiment, the RNAi polynucleotide is an siRNA.

In various embodiments, a target RNA (also referred to herein as a target gene) comprises a polynucleotide encoding a polypeptide. In certain embodiments, the target RNA is the polynucleotide region encoding the polypeptide. In other embodiments, the polynucleotide region comprises a regulatory sequence, for example sequences that regulate replication, transcription, RNA maturation, or translation or other processes important to expression of the polypeptide. In specific embodiments, regulatory sequences include, but are not limited to, 3′ untranslated regions (UTRs), 5′ UTRs, intron splice donor or splice acceptors, or other regulatory motifs. In still other embodiments, the target RNA comprises both the region encoding the polypeptide and the region operably linked thereto that regulates expression. In a particular embodiment, the target RNA is processed and consists essentially of exon sequences.

Any gene being expressed in a cell can be targeted. In a particular embodiment, a target gene is one involved in or associated with the progression of cellular activities important to disease or of particular interest as a research object. In various embodiments, the RNAi polynucleotide prevents the expression of a protein whose activity is necessary for the maintenance of a certain disease state, such as, for example, an oncogene. In cases where the oncogene is a mutated from of a gene, then the RNAi polynucleotide may preferentially prevent the expression of the mutant oncogene and not the wild-type protein.

In designing RNAi polynucleotides, there are several factors to be considered, such as the nature of the RNAi polynucleotide, the durability of the silencing effect, and the choice of delivery system. For example, the RNAi process is homology dependent. In a variety of embodiments, the RNAi polynucleotide sequences must be selected to maximize gene specificity, while minimizing the possibility of cross-interference between homologous, but not gene-specific sequences. In a series of embodiments using siRNA as the RNAi polynucleotide, the siRNA sequence exhibits greater than 80%, 85%, 90%, 95%, 98%, or even 100% identity to the target gene to be inhibited. Sequences less than about 80% identical to the target gene are substantially less effective. Thus, the greater homology between the siRNA and the gene to be inhibited, the less likely expression of unrelated genes will be affected. In other various embodiments, properties of the RNAi polynucleotides, such as stability, can be modified or altered. Examples of polynucleotide modifications are described in greater detail in Section I.

In certain embodiment of the invention, the presence of free radical scavengers allows for a greater possible voltage to be used during electroporation. In specific embodiments, the greater voltage allows for improved transfection efficiency and/or a functional improvement for electroporated siRNA molecules. In a particular embodiment, the electroporated siRNA molecules demonstrate increased RNAi potency, e.g., gene expression knockdown.

### VI. CRISPR Mediated Gene Editing

Clustered Regularly Interspaced Short Palindromic Repeats (CRISPR) systems refer to systems useful for genome editing including, but limited to, excision of genes, mutating of genes (e.g., introduction of point mutations, frame shift mutations, and other mutations that alter expression of a gene of interest), incorporation of exogenous gene elements (e.g., introduction of exogenous coding regions, such as affinity tags, fluorescent tags, and other exogenous markers), and editing via homology-directed repair (HDR). CRISPR systems, in general, use a CRISPR family enzyme (e.g., Cas9) and a guide RNA (gRNA) to direct nuclease activity (i.e., cutting of DNA) in a target specific manner within a genome. Polynucleotides useful in CRISPR systems are known to those skilled in the art and include, but are not limited to, a guide RNA (gRNA), a CRISPR RNA (crRNA), a trans-activating CRISPR RNA (tracrRNA), a single-guide crRNA and tracrRNA fusion (sgRNA), a polynucleotide encoding a CRISPR family enzyme, a CRISPR system expression vector (e.g., a vector encoding a CRISPR family enzyme, a gRNA, a crRNA, a tracrRNA, a sgRNA, or combinations thereof), or combinations thereof. CRISPR systems are described in more detail in M. Adli. (“The CRISPR tool kit for genome editing and beyond” Nature Communications; volume 9 (2018), Article number: 1911), herein incorporated by reference for all that it teaches.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Methods

### Anion Exchange Chromatography

Anion exchange chromatography (AEX) was carried out on an Agilent 1100 HPLC using a DNAPAC™ PA200 column (Thermo Scientific). The column was equilibrated in 50 mM Tris pH 7.4, and ˜20 ng of each siRNA sample was prepared in a 100 μl HPLC vial for injection. The sample was injected and subjected to a gradient of 50 mM Tris pH 7.4 (mobile phase A) and 50 mM Tris, 1 M NaCl, pH 7.4 (mobile phase B) according to Tables 1 and 2, below.

TABLE 1
Instrument method parameters

| | |
|---|---|
| Column compartment temperature | 40° C. |
| Sample compartment temperature | 5° C. |
| Flow rate | 0.5 mL/min |
| Run time | 20 min |
| UV detection¹ | 254 nm |
| Injection volume² | 100 μL but may be sample specific |
| High pressure limit | 300 psi |

TABLE 2
Gradient parameters

| Time | Flow Rate | % Mobile | % Mobile |
|---|---|---|---|
| (min) | (mL/min) | Phase A | Phase B |
| 0 | 0.5 | 65 | 35 |
| 3 | 0.5 | 65 | 35 |
| 10.5 | 0.5 | 40 | 60 |
| 11.5 | 0.5 | 0 | 100 |
| 14.5 | 0.5 | 0 | 100 |
| 15 | 0.5 | 65 | 35 |
| 20 | 0.5 | 65 | 35 |

### Peptide-Nucleic Acid Anion Exchange Chromatography for Exosome Loading Quantitation

A modified version of the AEX protocol described above was used to quantitate the relative amount of siRNA in electroporated exosomes. Exosome/siRNA electroporation products were prepared by combining 100 μL of Exosome/siRNA samples with 10 μL of lysis solution (100 nM complementary peptide-nucleic acid [PNA] conjugated to Atto 520, 1.1% Triton in water). Samples were vortexed and incubated at 95° C. for 15 minutes. Samples were cooled to room temperature and transferred into an HPLC vial for injection. Samples were injected on a DNAPAC™ PA200 column (Thermo Scientific) and subjected to a gradient of 25 mM Tris pH 8, 1 mM EDTA, 50% Acetonitrile (Mobile Phase A) and 25 mM Tris pH 8, 1 mM EDTA, 0.8 M NaClO4, 50% Acetonitrile (Mobile Phase B) according to Tables 3 and 4, below.

TABLE 3
Instrument method parameters

| | |
|---|---|
| Column compartment temperature | 50° C. |
| Sample compartment temperature | 5° C. |
| Flow rate | 0.5 mL/min |
| Run time | 17 min |
| FL detection¹ | Excitation 520 nm; emission 550 nm |
| FLD Gain² | PMT gain = 15 |
| Injection volume³ | 100 μL but may be sample specific |
| High pressure limit | 300 psi |

TABLE 4
Gradient parameters

| Time | Flow Rate | % Mobile | % Mobile |
|---|---|---|---|
| (min) | (mL/min) | Phase A | Phase B |
| 0 | 0.5 | 95 | 5 |
| 2 | 0.5 | 95 | 5 |
| 8 | 0.5 | 20 | 80 |
| 8.5 | 0.5 | 50 | 50 |
| 10.5 | 0.5 | 50 | 50 |
| 11 | 0.5 | 5 | 95 |
| 17 | 0.5 | 5 | 95 |

### Apoptosis Quantification

Panc-1 cells (100,000 cells per well) were seeded in a 6 well plate. The following day, cells were washed and treated with one of several conditions comprising siRNA and/or exosomes in low serum media. For a positive control, cells were transfected with XD-08318 ant-KRAS G12D siRNA using Lipofectamine® RNAiMax (ThermoFisher) according the manufacturer's specifications. 72 hours post treatment, cells were trypsinzed and apoptosis was measured according to manufacturer's specifications (Abeam, catalog no. ab14085). The samples were measured by using the Sony Spectral Cell Analyzer SA3800. All control samples were run side by side with experimental samples. Each sample was run in technical triplicates.

### Example 1: Anion Exchange Spectra Demonstrate Electroporation Induced Changes in siRNA with Altered Nucleotides

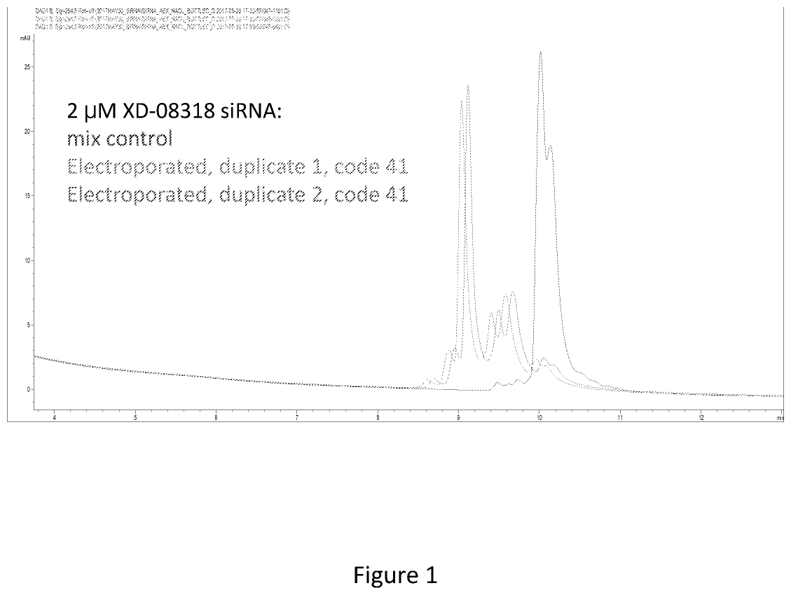

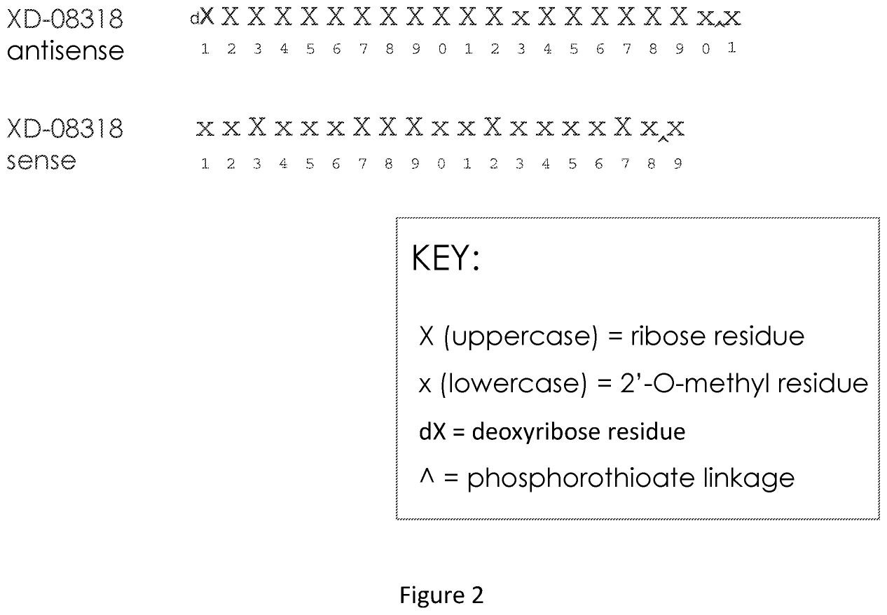

Electroporation is a common method for introducing nucleic acids into cells and other lipid structures such as exosomes. As an analytical method, siRNAs were analyzed by anion exchange chromatography (AEX), as described above, before and after electroporation. Surprisingly, a synthetic siRNA targeting KRAS G12D, XD-08318, underwent a spectral shift after electroporation as measured by AEX (FIG. 1). A 2 μM solution of unelectroporated XD-08318 (mix control) eluted from the AEX column as a major peak at roughly 10 minutes. Duplicate samples of a 2 μM mixture of XD-08318 electroporated on a MaxCyte® GT using pulse code 41 eluted from the AEX column as several distinct species, indicating a change to the confirmation or structure of the siRNA. XD-08318 is a modified siRNA that contains a 5′ terminal deoxyribose residue on the antisense strand, a combination of natural ribose and synthetic 2′-O-methyl ribose residues, and phosphorothioate linkages at the 3′ ends of each strand (FIG. 2).

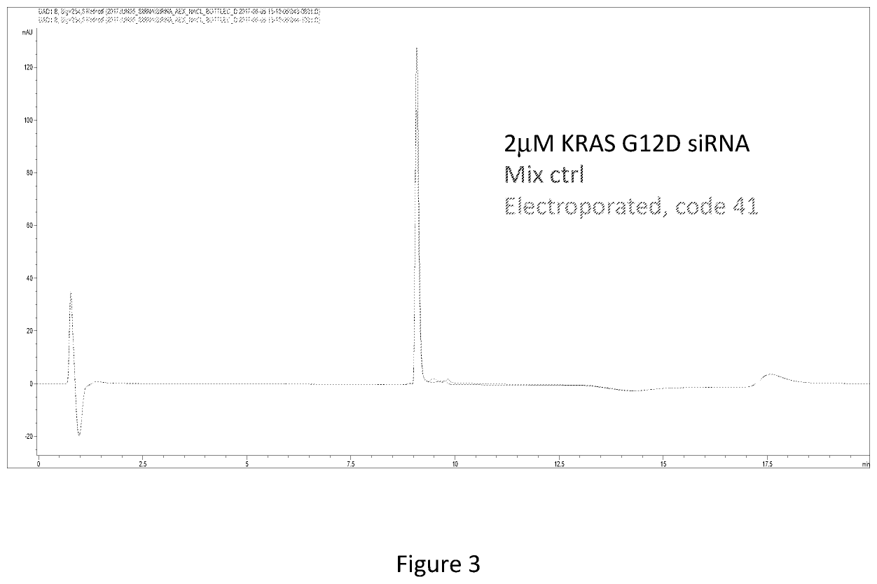

An unmodified siRNA encoding KRAS G12D was electroporated under similar conditions and analyzed by AEX. As shown in FIG. 3, there was no change to the spectral properties of the KRAS G12D siRNA, indicating that the changes observed in FIG. 1 are likely due to the chemical modifications of XD-08318.

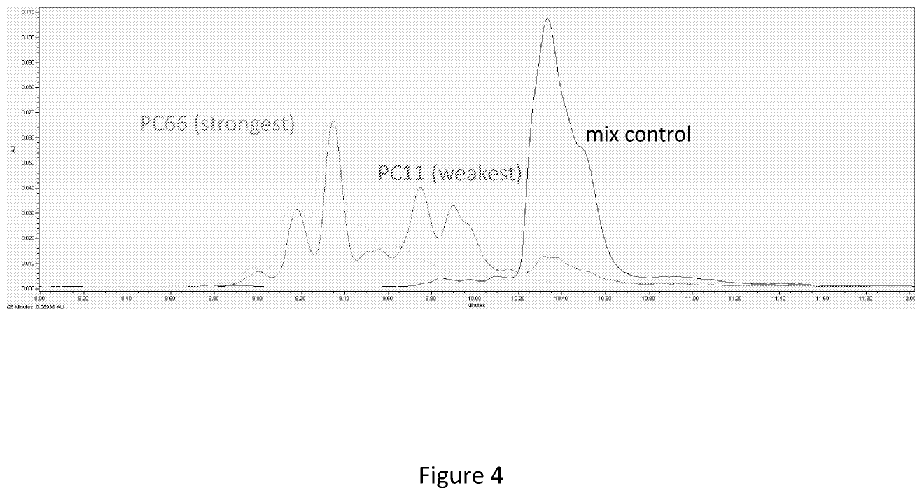

Modified synthetic RNA was electroporated using several electroporation conditions with varying electrical field conditions. Using a MaxCyte® GT at pulse code 66 showed complete loss of the peak observed in the mix control condition. Using pulse code 11, a weaker electrical field condition, the spectral shift was incomplete, suggesting that electroporation strength is correlated with the extent of siRNA change (FIG. 4).

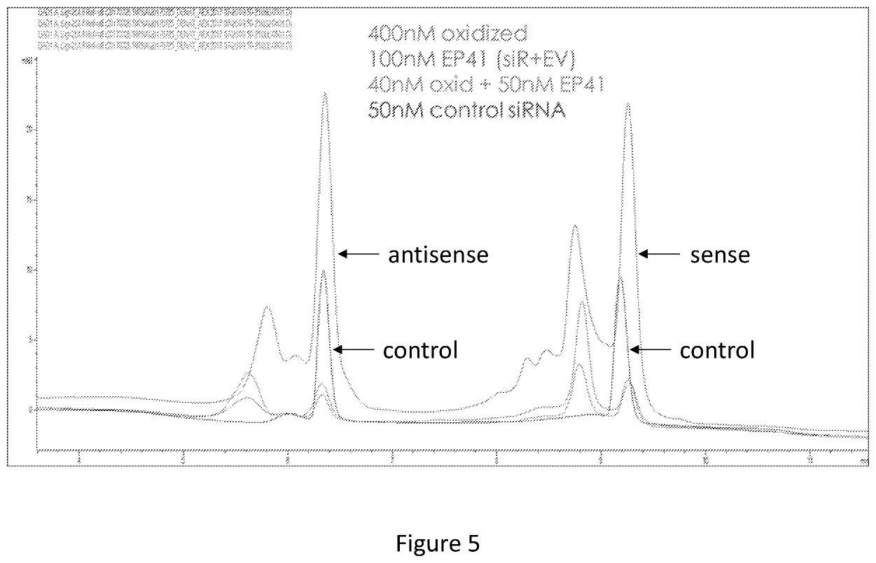

Electroporation was compared to forced oxidation using hydrogen peroxide for each of the two strands of the siRNA duplex. As shown in FIG. 5, the sense and antisense strands of untreated XD-08318 (control siRNA) eluted from the AEX column as single peaks. When each of the strands was exposed to either electroporation with pulse code 41 (EP41), hydrogen peroxide (oxidized), or electroporation in the presence of hydrogen peroxide (oxid+EP41), the spectral shifts were similar, suggesting that electroporation induces oxidation of the synthetic RNA (FIG. 5). The chemical modifications of XD-08318 suggest that the oxidation occurs at the phosphorothioate residues at the 3′ terminal nucleotide linkage.

### Example 2: Anion Exchange Spectra Demonstrate Reduced Eletroporation Induced Affects in the Presence of Antioxidants

Electroporation-induced oxidation of synthetic RNA could result in loss of terminal nucleotides and alter the targeting ability of the siRNA. Furthermore, many synthetic nucleic acid sequences are modified with phosphorothioate linkages to stabilize against RNase degradation. If electroporation can oxidize phosphorothioate linkages, then synthetic RNAs may be susceptible to substantial degradation and reduced potency.

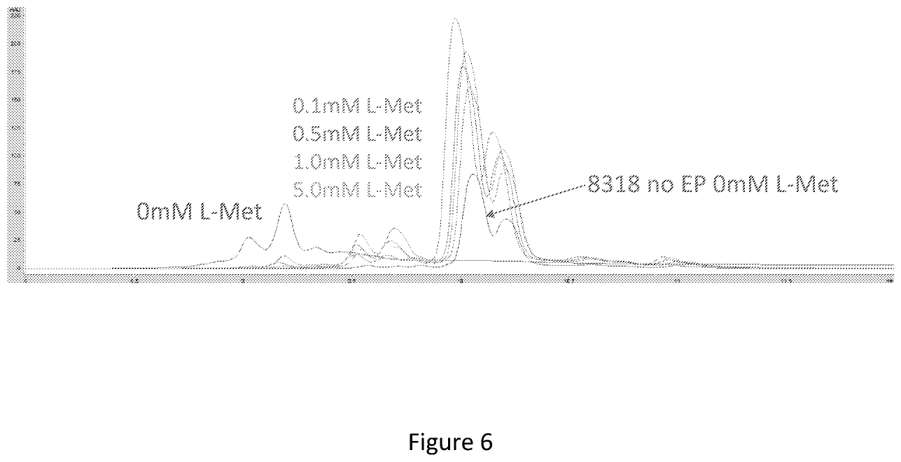

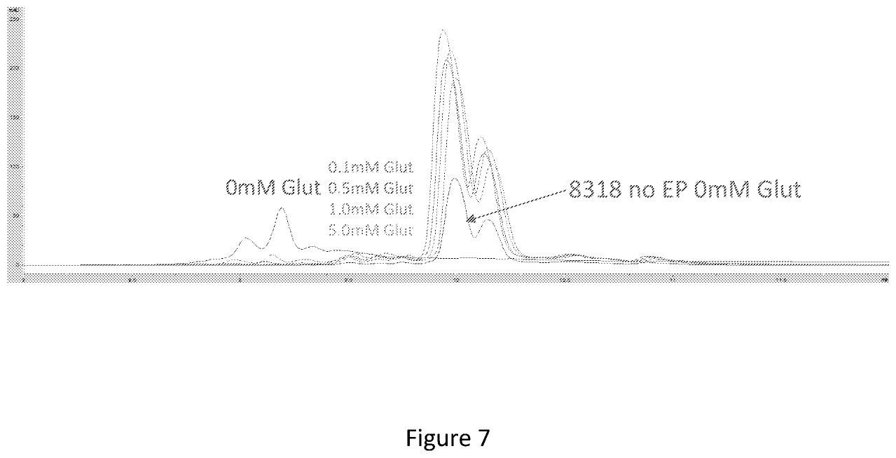

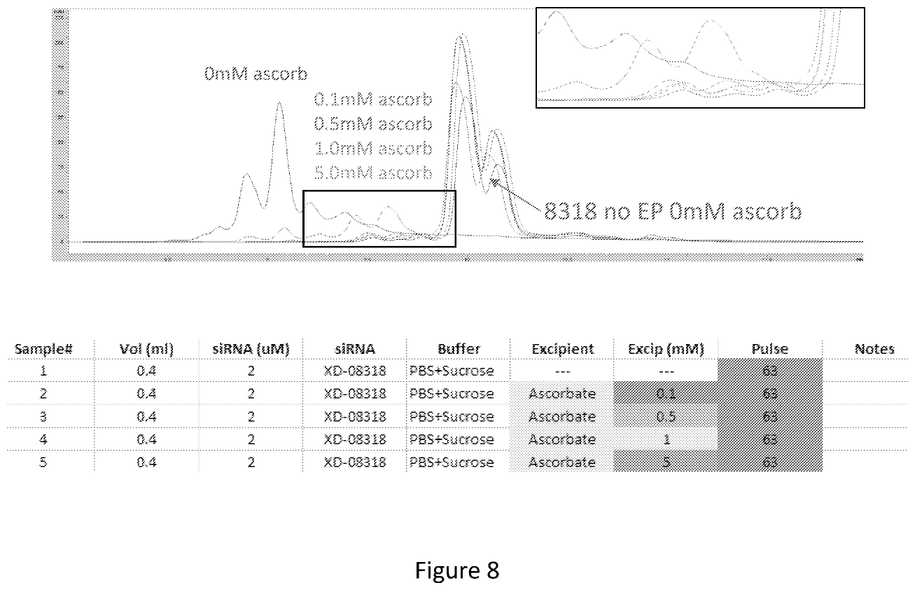

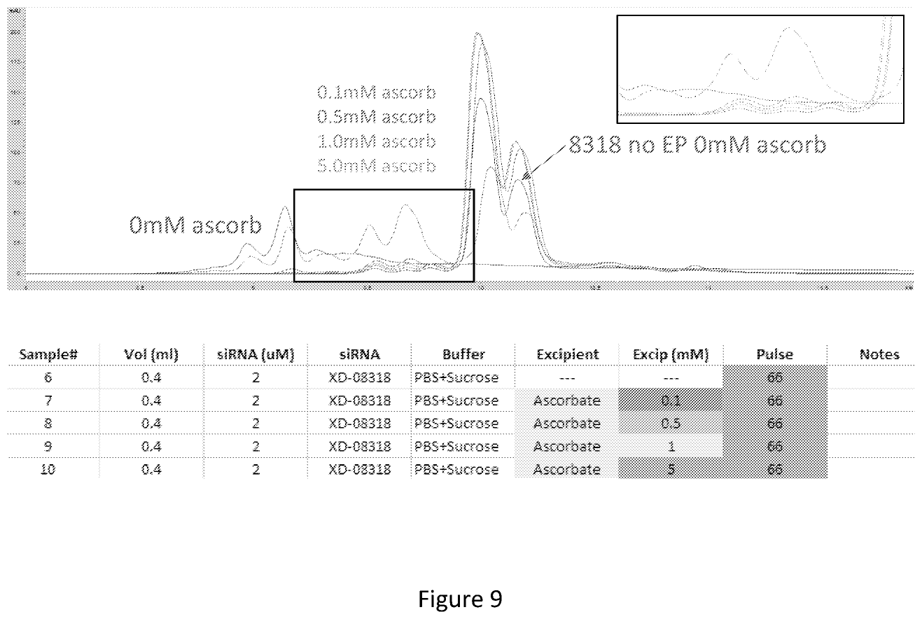

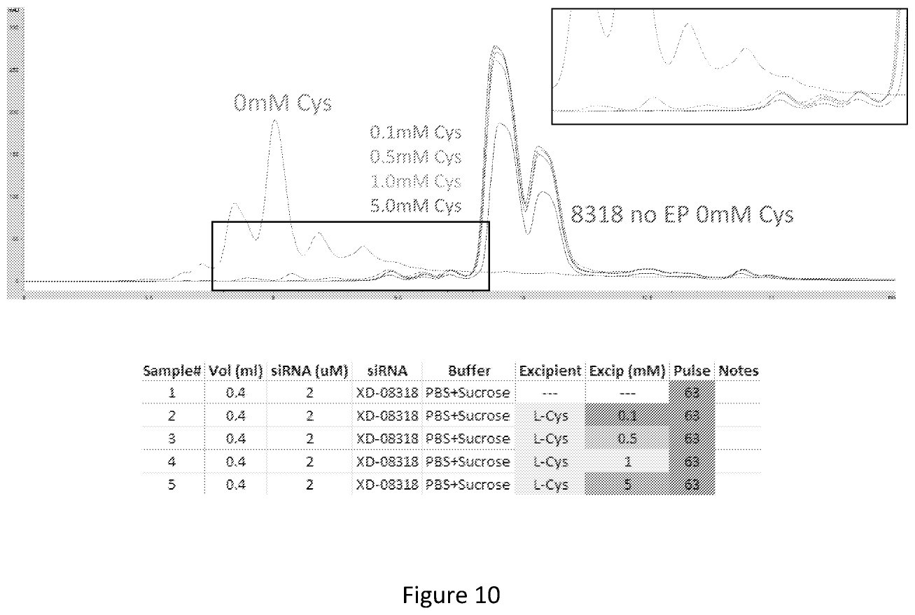

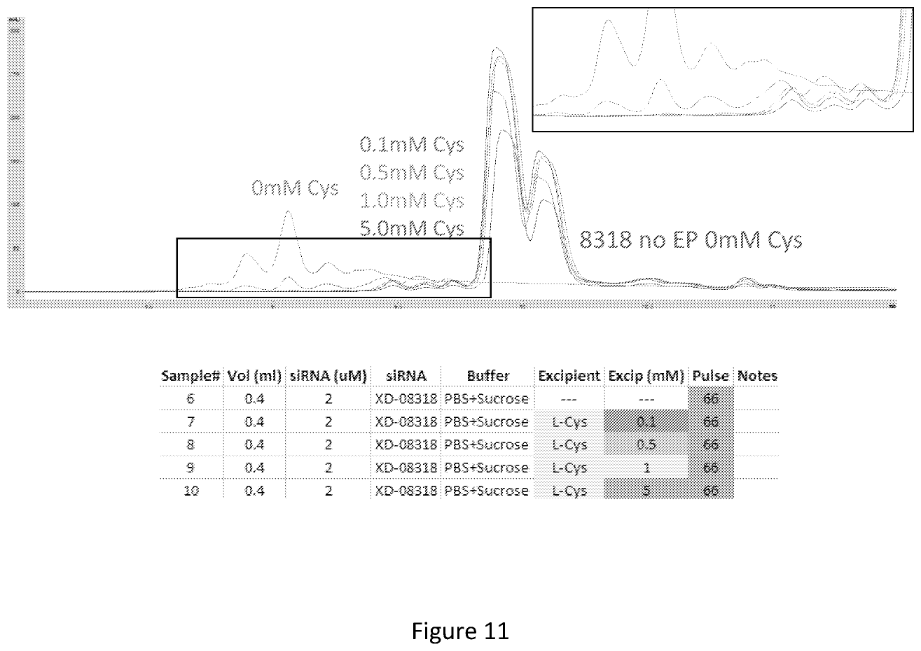

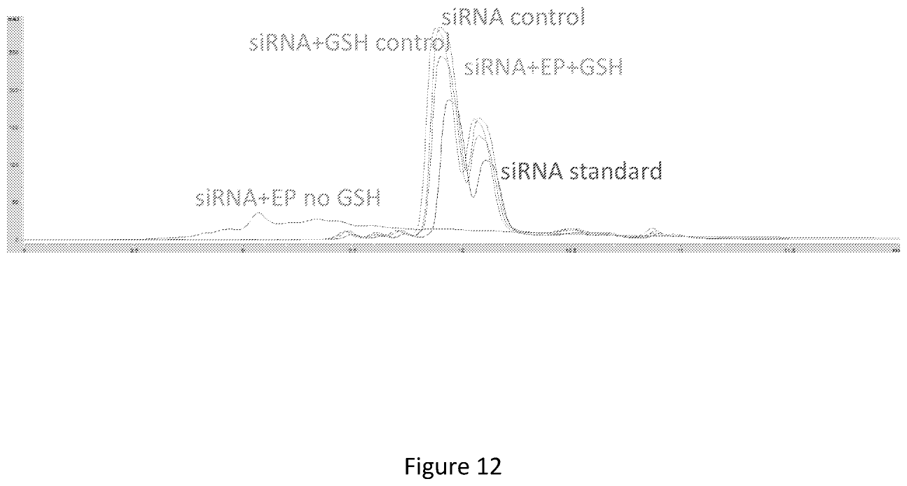

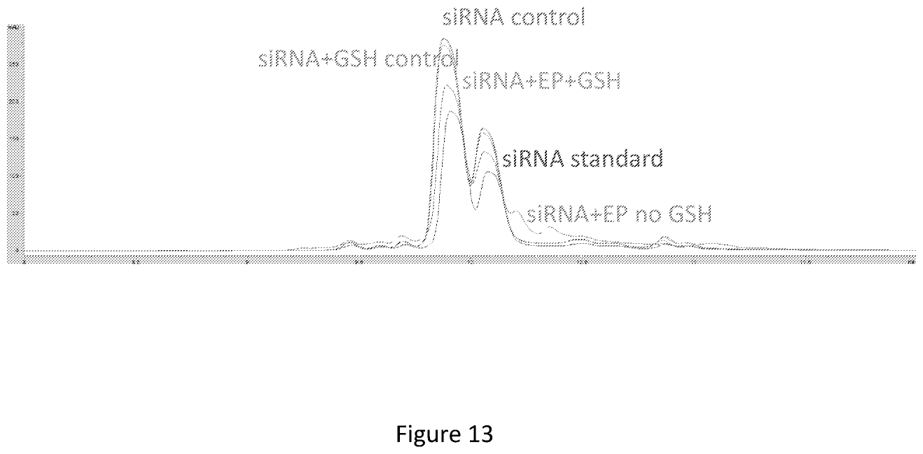

Synthetic RNA XD-08318 was electroporated in the presence of several free radical scavengers and reducing agents as excipients. In all cases tested, the addition of a free radical scavenger or reducing agent mitigated or prevented oxidation-induced changes to the siRNA. L-Methionine (FIG. 6) and Glutathione (FIG. 7) at strong pulse code 66 (high field strength, high frequency) prevented the spectral shift of XD-08318. Additionally, ascorbate or L-cysteine at an intermediate pulse code 63 (high field strength, low frequency) (FIGS. 8 and 10) or a strong pulse code 66 (FIGS. 9 and 11) also prevented the spectral shift of XD-08318. Furthermore, glutathione (GSH) was sufficient to prevent oxidation of XD-08318 after electroporation using two different electroporation devices. XD-08318 siRNA was electroporated using the Neon Transfection System (ThermoFisher) at 2000 V, 5 ms per pulse, and 8 pulses (FIG. 12) or the Gene Pulser XCell (Bio-Rad) at 800 V, 5 ms per pulse, and 2 pulses (FIG. 13), and in both cases GSH was able to prevent a change in the AEX profile of the siRNA. These results suggest that electroporation-induced oxidation is a general effect, and that the addition of a number of simple excipients is sufficient to prevent the degradation of phosphorothioate-containing RNA during electroporation.

### Example 3: Addition of Antioxidants Improves Transfection Efficiency and siRNA Efficacy

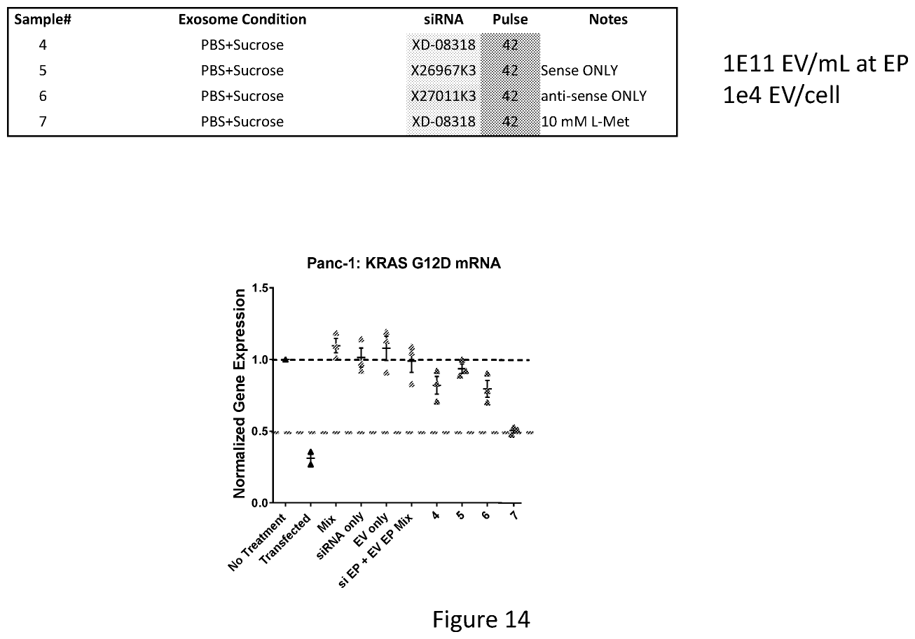

XD-08318 was loaded into exosomes by electroporation in the presence or absence of free radical scavengers. As a control, XD-08318 was transfected into the human pancreatic cancer cell line Panc-1, which is heterozygous for the G12D mutant transcript of KRAS. Transfection resulted in ˜75% knockdown of the KRAS G12D transcript (FIG. 14). As controls, in the absence of any transfection reagent, Panc-1 cells were incubated with a mixture of the siRNA and exosomes without electroporation (mix), siRNA only, exosomes alone (EV only), or in the presence of siRNA and exosomes that were electroporated individually (si EP+EV EP Mix). None of these conditions resulted in a decrease in KRAS G12D expression. Next, XD-08318 was loaded into exosomes by electroporation at pulse code 42 (sample 4) and incubated with cells, resulting in a modest ˜30% repression of the target transcript. Neither the sense strand nor the antisense strand alone electroporated into exosomes increased target knockdown (samples 5 and 6, respectively). In contrast, XD-08318 electroporated into exosomes in the presence of 10 mM L-methionine enhance target knockdown to ˜50%, indicating that the presence of a free radical scavenger excipient during electroporation results in more potent siRNA-loaded exosomes.

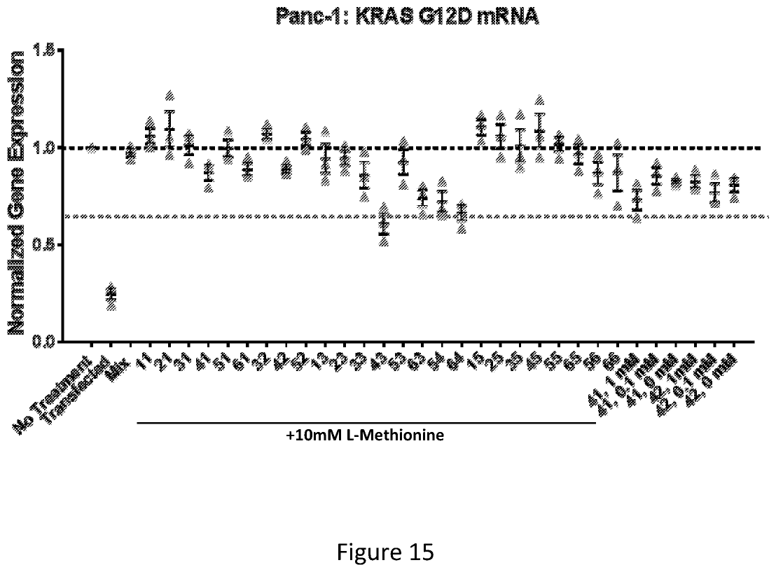

The results in FIG. 14 suggest that electroporation-induced oxidation of phosphorothioate-containing siRNAs can be reduced by the addition of a single additive, and thus may permit the use of stronger electrical fields in loading exosomes. FIG. 15 shows the results of KRAS G12D knockdown with siRNA loaded exosomes using 25 different pulse codes on a MaxCyte® GT. In the presence of L-Methionine, stronger pulse codes were able to be accessed for loading exosomes without compromising the repressive ability of the siRNA, resulting in potent knockdown of KRAS G12D at strong pulse codes.

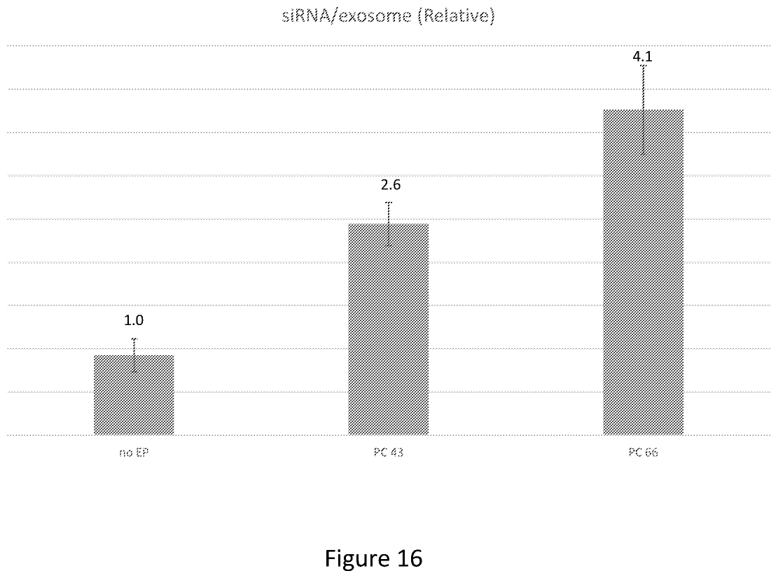

Exosome loading was measured using the PNA complementarity AEX method described above. As shown in FIG. 16, exosomes mixed with XD-08318 led to low levels of siRNA incorporation. When the mixture was electroporated in the presence of glutathione using intermediate pulse code 43, there was a 2.6-fold increase in loading. Using the strong pulse code 66, there was a 4.1-fold increase in loading compared to the mix control. These results demonstrate the benefit of accessing stronger electrical conditions to allow for enhanced loading efficiency, which requires the addition of antioxidants to protect phosphorothioate residues.

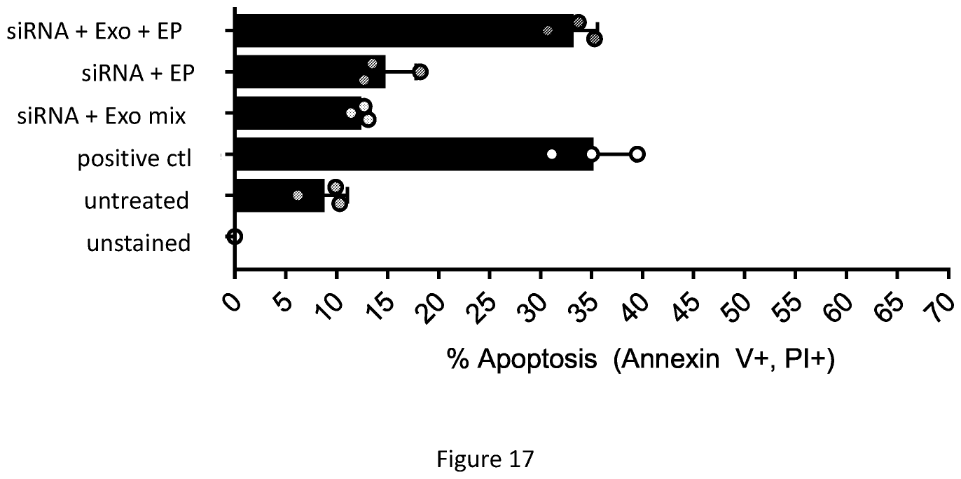

Exosomes electroporated with pulse code 66 were tested in their ability to induce apoptosis in a human pancreatic cancer cell line. Panc-1 cancer cells express oncogenic KRAS G12D and become apoptotic upon inhibition of the KRAS G12D transcript (Nature 546, 498-503 (2017)). As shown in FIG. 17, <10% of cultured Panc-1 were apoptotic as measured by Annexin V and propidium iodide staining. In contrast, transfecting Panc-1 cells with XD-08318 using Lipofectamine induced apoptosis to ˜35% of the cell population. Neither XD-08318 siRNA mixed with exosomes nor free siRNA electroporated in the presence of GSH at pulse code 66 induced apoptosis in Panc-1 cells. In contrast, exosomes electroporated in the presence of GSH and XD-08318 were able to induce apoptosis to a level similar to that of the positive control. These results confirm successful loading of exosomes and subsequent transfer of a phosphorothioate-containing siRNA, which could be enhanced by incubation with an anti-oxidizing agent.

What is claimed is:

1. A method of reducing nucleotide oxidation during electroporation, the method comprising the steps of:
1) providing a composition comprising a) a polynucleotide, wherein the polynucleotide comprises a nucleotide alteration, b) a free radical scavenger, and c) a recipient entity; and
2) electroporating the composition, wherein the free radical scavenger reduces electroporation-induced oxidation of the nucleotide alteration.

2. The method of claim 1, wherein the polynucleotide comprises RNA.

3. The method of claim 2, wherein the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof.

4. The method of claim 2, wherein the RNA is an siRNA.

5. The method of claim 3, wherein the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

6. The method of claim 1, wherein the polynucleotide comprises DNA.

7. The method of claim 6, wherein the DNA is selected from the group consisting of: circular plasmids, linear plasmids, vectors, single-stranded DNA, single-stranded oligonucleotides, double-stranded oligonucleotides, a CRISPR system expression vector, and combinations thereof.

8. The method of claim 7, wherein the CRISPR system expression vector encodes a CRISPR family enzyme, a gRNA, a crRNA, a tracrRNA, a sgRNA, and combinations thereof.

9. The method of claim 1, wherein the polynucleotide comprises a non-natural nucleic acid.

10. The method of claim 9, wherein the non-natural nucleic acid is a morpholino.

11. The method of any of claims 1-10, wherein the nucleotide alteration comprises a phosphorothioate internucleotide linkage.

12. The method of any of claims 1-11, wherein the free radical scavenger is a reducing agent.

13. The method of claim 12, wherein the reducing agent is selected from the group consisting of: L-Methionine, glutathione, L-cysteine, and ascorbic acid, and combinations thereof.

14. The method of claim 13, wherein the reducing agent is glutathione.

15. The method of any of claims 1-14, wherein the concentration of the free radical scavenger is between 0.1 mM to 100 mM.

16. The method of any of claims 1-15, wherein the recipient entity is a lipid-based entity.

17. The method of claim 16, wherein the lipid-based entity is selected from the group consisting of: a cell, a vesicle, a tissue, and a lipid-based nanoparticle.

18. The method of claim 17, wherein the lipid-based nanoparticle is selected from the group consisting of: a unilamellar liposome, a multilamellar liposome, a nanovesicle, and a lipid preparation.

19. The method of claim 17, wherein the vesicle is an extracellular vesicle.

20. The method of claim 19, wherein the extracellular vesicle is an exosome.

21. The method of claim 17, wherein the cell is selected from a eukaryotic cell or a prokaryotic cell.

22. The method of claim 21, wherein the eukaryotic cell is selected from the group consisting of: an animal cell, a fungal cell, and a plant cell.

23. The method of claim 22, wherein the animal cell is selected from a vertebrate cell or an invertebrate cell.

24. The method of claim 23, wherein the vertebrate cell is a mammalian cell.

25. The method of claim 24, wherein the mammalian cell is a human cell.

26. The method of any of claims 23-25, wherein the cell is selected from the group consisting of: a stem cell, an immune cell, an erythrocyte, a cancer cell, a cultured cell, an immortalized cell, and an isolated cell, and combinations thereof.

27. The method of claim 26, wherein the immune cell is selected from the group consisting of: a T cell, a B cell, a macrophage, and a dendritic cell.

28. The method of claim 22, wherein the fungal cell is a yeast cell.

29. The method of claim 21, wherein the prokaryotic cell is a bacterial cell.

30. The method of any of claims 1-15, wherein the recipient entity is a non-lipid entity.

31. The method of claim 30, wherein the non-lipid entity is a non-lipid nanostructure.

32. The method of any of claims 1-31, wherein the electroporating step is performed in vitro, in vivo, or ex vivo.

33. The method of any of claims 1-32, wherein the reduction in oxidation is determined through analyzing a molecular profile of the polynucleotide.

34. The method of claim 33, wherein the molecular profile is an anion exchange high-performance liquid chromatography (AEX-HPLC) chromatogram.

35. The method of claim 33, wherein the molecular profile is an ion-pairing reversed-phase chromatography (IPRP-HPLC) chromatogram.

36. The method of claim 33, wherein the molecular profile is a mass spectrometry spectrum.

37. The method of any of claims 33-36, wherein the molecular profile of the polynucleotide is shifted toward an unelectroporated polynucleotide relative to a polynucleotide electroporated in the absence of the free radical scavenger.

38. The method of any of claims 1-37, wherein the electroporating step comprises a voltage level higher than a viable electroporation voltage level in the absence of the free radical scavenger.

39. The method of claim 38, wherein the polynucleotide demonstrates a functional improvement at the voltage level.

40. The method of claim 39, wherein the functional improvement is an increased activity of the polynucleotide.

41. The method of claim 40, wherein the increased activity of the polynucleotide is an increase in RNA interference.

42. The method of claim 40, wherein the increased activity of the polynucleotide is an increase in CRISPR mediated gene editing.

43. A method of enhancing transfection efficiency, comprising the steps of:
1) providing a composition comprising a) a polynucleotide, wherein the polynucleotide comprises a nucleotide alteration, b) a free radical scavenger, and c) a recipient entity; and
2) electroporating the composition, wherein the free radical scavenger reduces electroporation-induced oxidation of the electroporated polynucleotide.

44. The method of claim 43, wherein the polynucleotide comprises RNA.

45. The method of claim 44, wherein the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof.

46. The method of claim 44, wherein the RNA is an siRNA.

47. The method of claim 45, wherein the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

48. The method of claim 43, wherein the polynucleotide comprises DNA.

49. The method of claim 48, wherein the DNA is selected from the group consisting of: circular plasmids, linear plasmids, vectors, single-stranded DNA, single-stranded oligonucleotides, double-stranded oligonucleotides, a CRISPR system expression vector, and combinations thereof.

50. The method of claim 49, wherein the CRISPR system expression vector encodes a CRISPR family enzyme, a gRNA, a crRNA, a tracrRNA, a sgRNA, and combinations thereof.

51. The method of claim 43, wherein the polynucleotide comprises a non-natural nucleic acid.

52. The method of claim 51, wherein the non-natural nucleic acid is a morpholino.

53. The method of any of claims 43-52, wherein the nucleotide alteration comprises a phosphorothioate internucleotide linkage.

54. The method of any of claims 43-53, wherein the free radical scavenger is a reducing agent.

55. The method of claim 54, wherein the reducing agent is selected from the group consisting of: L-Methionine, glutathione, L-cysteine, and ascorbic acid, and combinations thereof.

56. The method of claim 55, wherein the reducing agent is glutathione.

57. The method any of claims 43-56, wherein the concentration of the free radical scavenger is between 0.1 mM to 100 mM.

58. The method of any of claims 43-57, wherein the recipient entity is a lipid-based entity.

59. The method of claim 58, wherein the lipid-based entity is selected from the group consisting of: a cell, a vesicle, a tissue, and a lipid-based nanoparticle.

60. The method of claim 59, wherein the lipid-based nanoparticle is selected from the group consisting of: a unilamellar liposome, a multilamellar liposome, a nanovesicle, and a lipid preparation.

61. The method of claim 59, wherein the vesicle is an extracellular vesicle.

62. The method of claim 61, wherein the extracellular vesicle is an exosome.

63. The method of claim 59, wherein the cell is selected from a eukaryotic cell or a prokaryotic cell.

64. The method of claim 63, wherein the eukaryotic cell is selected from the group consisting of: an animal cell, a fungal cell, and a plant cell.

65. The method of claim 64, wherein the animal cell is selected from a vertebrate cell or an invertebrate cell.

66. The method of claim 65, wherein the vertebrate cell is a mammalian cell.

67. The method of claim 66, wherein the mammalian cell is a human cell.

68. The method of any of claims 65-67, wherein the cell is selected from the group consisting of: a stem cell, an immune cell, an erythrocyte, a cancer cell, a cultured cell, an immortalized cell, and an isolated cell, and combinations thereof.

69. The method of claim 68, wherein the immune cell is selected from the group consisting of: a T cell, a B cell, a macrophage, and a dendritic cell.

70. The method of claim 64, wherein the fungal cell is a yeast cell.

71. The method of claim 63, wherein the prokaryotic cell is a bacterial cell.

72. The method of any of claims 43-57, wherein the recipient entity is a non-lipid entity.

73. The method of claim 72, wherein the non-lipid entity is a non-lipid nanostructure.

74. The method of any of claims 43-73, wherein the electroporating step is performed in vitro, in vivo, or ex vivo.

75. The method of any of claims 43-74, wherein the reduction in oxidation is determined through analyzing a molecular profile of the polynucleotide.

76. The method of claim 75, wherein the molecular profile is an anion exchange high-performance liquid chromatography (AEX-HPLC) chromatogram.

77. The method of claim 75, wherein the molecular profile is an ion-pairing reversed-phase chromatography (IPRP-HPLC) chromatogram.

78. The method of claim 75, wherein the molecular profile is a mass spectrometry spectrum.

79. The method of any of claims 75-78, wherein the molecular profile of the polynucleotide is shifted toward an unelectroporated polynucleotide relative to a polynucleotide electroporated in the absence of the free radical scavenger.

80. The method of any of claims 43-79, wherein the electroporating step comprises a voltage level higher than a viable electroporation voltage level in the absence of the free radical scavenger.

81. The method of claim 80, wherein the polynucleotide demonstrates a functional improvement at the voltage level.

82. The method of claim 81, wherein the functional improvement is an increased activity of the polynucleotide.

83. The method of claim 82, wherein the increased activity of the polynucleotide is an increase in RNA interference.

84. The method of claim 82, wherein the increased activity of the polynucleotide is an increase in CRISPR mediated gene editing.

85. A composition for reducing nucleotide oxidation during electroporation, the composition comprising a) a polynucleotide, wherein the polynucleotide comprises a nucleotide alteration, b) a free radical scavenger, and c) a recipient entity.

86. The composition of claim 85, wherein the polynucleotide comprises RNA.

87. The composition of claim 86, wherein the RNA is selected from the group consisting of: siRNAs, miRNAs, antisense oligonucleotides, shRNAs, double-stranded RNAs, RNA oligonucleotides, mRNAs, a Clustered Regularly Interspaced Short Palindromic Repeats (CRISPR) system RNA, and combinations thereof.

88. The composition of claim 86, wherein the RNA is an siRNA.

89. The composition of claim 87, wherein the CRISPR system RNA is selected from the group consisting of: a guide RNA (gRNA), a CRISPR RNA (crRNA), a trans-activating CRISPR RNA (tracrRNA), and a single-guide crRNA and tracrRNA fusion (sgRNA), and combinations thereof.

90. The composition of claim 85, wherein the polynucleotide comprises DNA.

91. The composition of claim 90, wherein the DNA is selected from the group consisting of: circular plasmids, linear plasmids, vectors, single-stranded DNA, single-stranded oligonucleotides, double-stranded oligonucleotides, a CRISPR system expression vector, and combinations thereof.

92. The composition of claim 91, wherein the CRISPR system expression vector encodes a CRISPR family enzyme, a gRNA, a crRNA, a tracrRNA, a sgRNA, and combinations thereof.

93. The composition of claim 85, wherein the polynucleotide comprises a non-natural nucleic acid.

94. The composition of claim 93, wherein the non-natural nucleic acid is a morpholino.

95. The method of any of claims 85-94, wherein the nucleotide alteration comprises a phosphorothioate internucleotide linkage.

96. The method of any of claims 85-95, wherein the free radical scavenger is a reducing agent.

97. The composition of claim 96, wherein the reducing agent is selected from the group consisting of: L-Methionine, glutathione, L-cysteine, and ascorbic acid, and combinations thereof.

98. The composition of claim 97, wherein the reducing agent is glutathione.

99. The method of any of claims 85-98, wherein the concentration of the free radical scavenger is between 0.1 mM to 100 mM.

100. The method of any of claims 85-99, wherein the recipient entity is a lipid-based entity.

101. The composition of claim 100, wherein the lipid-based entity is selected from the group consisting of: a cell, a vesicle, a tissue, and a lipid-based nanoparticle.

102. The composition of claim 101, wherein the lipid-based nanoparticle is selected from the group consisting of: a unilamellar liposome, a multilamellar liposome, a nanovesicle, and a lipid preparation.

103. The composition of claim 101, wherein the vesicle is an extracellular vesicle.

104. The composition of claim 103, wherein the extracellular vesicle is an exosome.

105. The composition of claim 101, wherein the cell is selected from a eukaryotic cell or a prokaryotic cell.

106. The composition of claim 105, wherein the eukaryotic cell is selected from the group consisting of: an animal cell, a fungal cell, and a plant cell.

107. The composition of claim 106, wherein the animal cell is selected from a vertebrate cell or an invertebrate cell.

108. The composition of claim 107, wherein the vertebrate cell is a mammalian cell.

109. The composition of claim 108, wherein the mammalian cell is a human cell.

110. The method of any of claims 107-109, wherein the cell is selected from the group consisting of: a stem cell, an immune cell, an erythrocyte, a cancer cell, a cultured cell, an immortalized cell, and an isolated cell, and combinations thereof.

111. The composition of claim 110, wherein the immune cell is selected from the group consisting of: a T cell, a B cell, a macrophage, and a dendritic cell.

112. The composition of claim 106, wherein the fungal cell is a yeast cell.

113. The composition of claim 105, wherein the prokaryotic cell is a bacterial cell.

114. The method of any of claims 85-99, wherein the recipient entity is a non-lipid entity.

115. The composition of claim 114, wherein the non-lipid entity is a non-lipid nanostructure.

116. A method of reducing nucleotide oxidation during electroporation, the method comprising the steps of:
1) providing a composition comprising the composition of any of claims 85-115; and
2) electroporating the composition, wherein the free radical scavenger reduces electroporation-induced oxidation of the nucleotide alteration.

117. A method of enhancing transfection efficiency, the method comprising the steps of:
1) providing a composition comprising the composition of any of claims 85-115; and
2) electroporating the composition, wherein the free radical scavenger reduces electroporation-induced oxidation of the electroporated polynucleotide.
